(19) United States
(12) Patent Application Publication
(10) Pub. No.: US 2025/0241800 A1
(43) Pub. Date: Jul. 31, 2025

(54) MENSTRUATION PAD WITH INTERGLUTEAL INSERT

(71) Applicant: Andrea Brown, Brooklyn, NY (US)

(72) Inventor: Andrea Brown, Brooklyn, NY (US)

(21) Appl. No.: 18/428,596

(22) Filed: Jan. 31, 2024

(51) Int. Cl. A61F 13/472; A61F 13/551; A61F 13/56

(52) U.S. Cl. CPC A61F 13/47227; A61F 13/5514; A61F 13/5616

(57) ABSTRACT

A menstruation pad for absorbing exudate from a user, including exudate which might otherwise flow into the intergluteal cleft of the user includes a base panel comprising a flexible material and an absorbent body mounted atop the base panel. A back insertion member is mounted to the absorbent body and is shaped to insert into the intergluteal cleft. Buttock engagement flaps are coupled to the base panel and are positioned on opposite sides of the back insertion member. The buttock engagement flaps are thin such that their outlines are minimized when viewed through the undergarment.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to menstruation pad and more particularly pertains to a new menstruation pad for absorbing exudate from a user, including exudate which might otherwise flow into the intergluteal cleft of the user.

### (2) Description of Related Art Including Information Disclosed Under 37 CFR 1.97 and 1.98

The prior art discloses various menstruation pads which have a portion which inserts into the intergluteal cleft of a user. However, such menstruation pads either do not have structure that abuts the user's buttocks to secure the menstruation pad in the intergluteal cleft or include absorbent material that adds definition to such structures, causing the menstruation pad to be move visible through garments worn by the user. The prior art fails to describe an apparatus which uses an absorbent insert that inserts into the intergluteal cleft of the user and which includes flaps to engage the user's buttocks while minimizing visibility through garments worn by the user.

### (h) BRIEF SUMMARY OF THE INVENTION

An embodiment of the disclosure meets the needs presented above by generally comprising a base panel which is elongated between a front end and a back end and comprises a flexible material. An absorbent body is mounted on a top surface of the base panel and is elongated parallel to the base panel. The absorbent body comprises an absorbent material and has a front portion, a back portion, and a middle portion. A back insertion member also comprises the absorbent material and is mounted atop the back portion of the absorbent body. The back insertion member is elongated and is aligned parallel to the absorbent body. The back insertion member is sized and configured to fit into an intergluteal cleft of a user. A pair of buttock engagement flaps is coupled to the base panel and positioned on opposite sides of the back insertion member. Each buttock engagement flap comprises the flexible material and has a thickness to minimize visibility of an outline of the buttock engagement flap through an undergarment worn by the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top view of a menstruation pad according to an embodiment of the disclosure.

FIG. 2 is a bottom view of an embodiment of the disclosure.

FIG. 3 is a back view of an embodiment of the disclosure.

FIG. 4 is a side view of an embodiment of the disclosure.

FIG. 5 is an in-use view of an embodiment of the disclosure.

### (j) DETAILED DESCRIPTION OF THE INVENTION

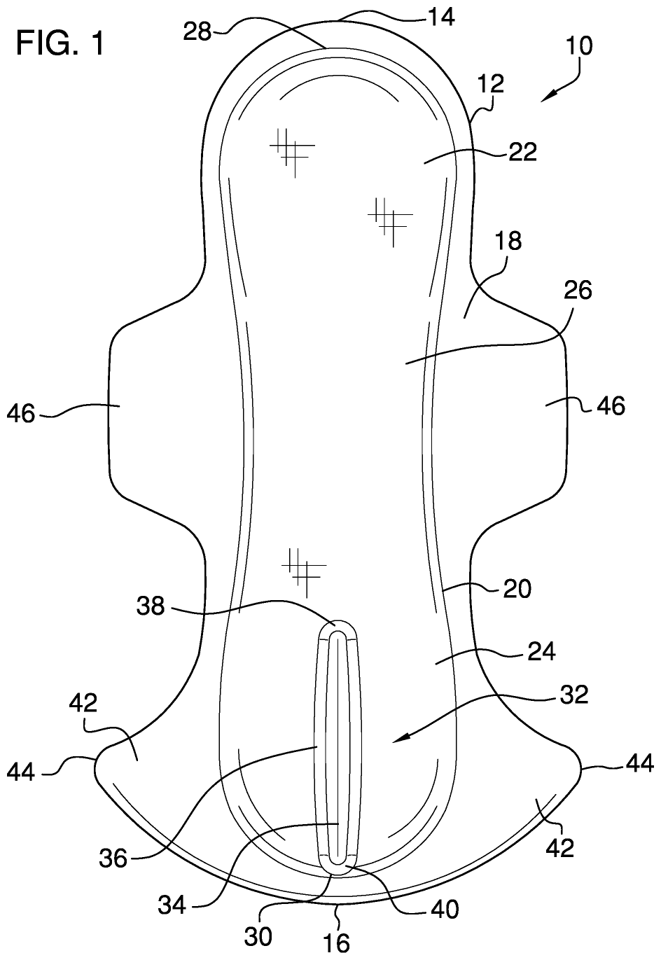

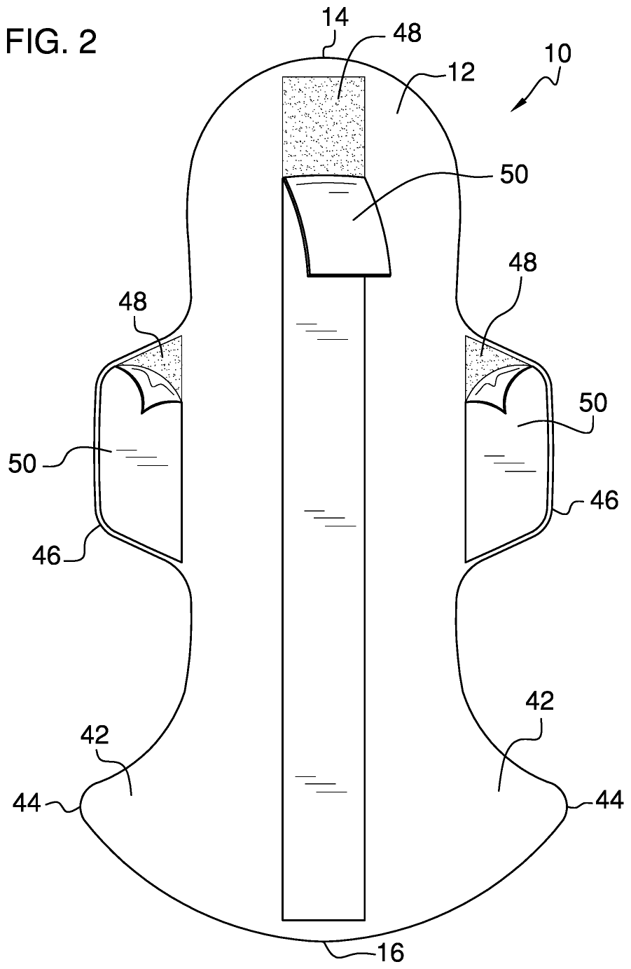

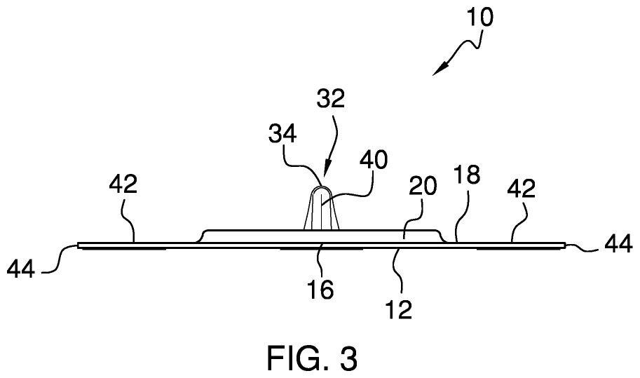

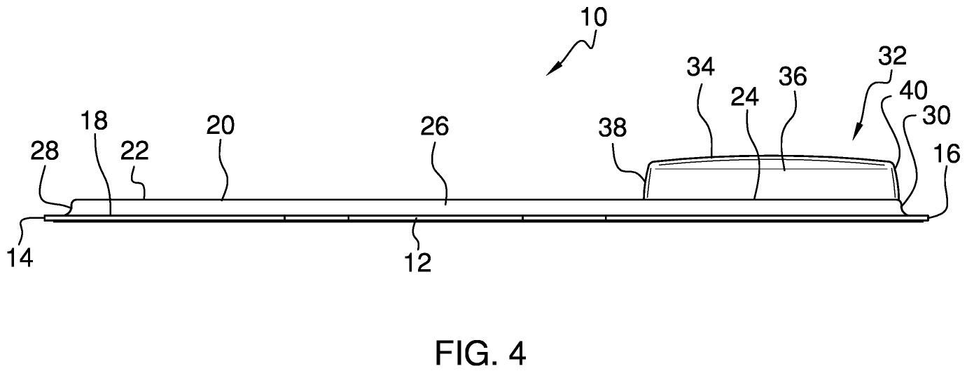

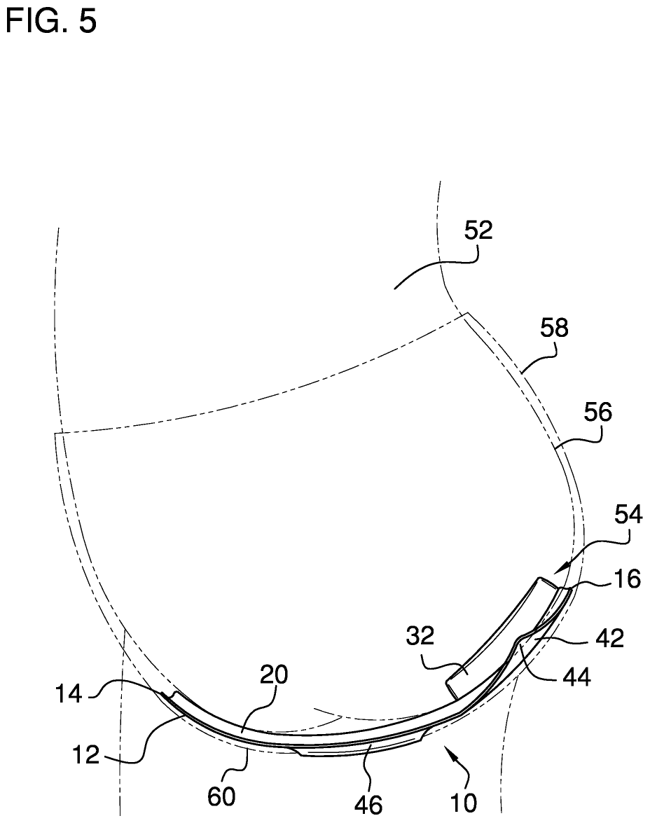

With reference now to the drawings, and in particular to FIGS. 1 through 5 thereof, a new menstruation pad embodying the principles and concepts of an embodiment of the disclosure and generally designated by the reference numeral 10 will be described.

As best illustrated in FIGS. 1 through 5, the menstruation pad 10 generally comprises a base panel 12 which is elongated between a front end 14 and a back end 16 and comprises a flexible material. An absorbent body 20 is mounted on a top surface 18 of the base panel 12 and is elongated in parallel to the base panel 12. The absorbent body 20 has a front portion 22, a back portion 24, and a middle portion 26. The absorbent body 20 narrows from each of the front portion 22 and the back portion 24 to the middle portion 26. A forward end 28 and a rearward end 30 of the absorbent body 20 are rounded. The absorbent body 20 comprises an absorbent material for absorbing exudate from a user 52 such as menstrual blood and other bodily fluids. The absorbent material may comprise cotton, other fibers, a sponge, or the like.

A back insertion member 32 also comprising the absorbent material is mounted atop and integrally formed with the back portion 24 of the absorbent body 20. The back insertion member 32 is elongated and is aligned parallel to the absorbent body 20. The back insertion member 32 is sized and configured to fit into an intergluteal cleft 54 of the user 52. The back insertion member 32 tapers from the absorbent body 20 to a distal edge 34 with respect to the absorbent body 20. The back insertion member 32 also tapers from a central section 36 of the back insertion member 32 to each of a frontmost end 38 and a backmost end 40 of the back insertion member 32.

A pair of buttock engagement flaps 42 is coupled to the base panel 12. The buttock engagement flaps 42 are positioned on opposite sides of the back insertion member 32. Each buttock engagement flap 42 comprises the flexible material and is integrally formed with the base panel 12. Each buttock engagement flap 42 has a thickness such that the buttock engagement flap 42 is configured to minimize visibility of an outline of the buttock engagement flap 42 through an undergarment 58 worn by the user 52. The buttock engagement flaps 42 are free of any absorbent material which would act to add unwanted thickness to the buttock engagement flaps 42, thereby adding definition to the outline of the buttock engagement flaps 42. Each buttock engagement flap 42 tapers away from the base panel 12 to a distal end 44 with respect to the base panel 12.

A pair of wings 46 is coupled to the base panel 12. The wings 46 are positioned on opposite sides of the middle portion 26 of the absorbent body 20. Each wing 46 comprises the flexible material and is integrally formed with the base panel 12. A plurality of adhesive layers 48 are provided and are configured to releasably attach to the undergarment 58. Each adhesive layer 48 is mounted on an associated one of the base panel 12 and either wing 46 of the pair of wings 46. Release liners 50 cover the adhesive layers 48 to prevent the adhesive layers 48 from attaching to objects before using the menstruation pad 10.

The flexible material forming the base panel 12, the buttock engagement flaps 42, and the wings 46 may comprise a fabric, a polymer sheet, or the like. The flexible material may also be water impermeable such that any fluids absorbed by the absorbent body 20 do not leak through the base panel 12, the buttock engagement flaps 42, or the wings 46. The menstruation pad 10 is sized such that the absorbent pad is positionable under the user's 52 vagina while the back insertion member 32 is placed in the user's 52 intergluteal cleft 54.

In use, the menstruation pad 10 is positioned with the absorbent body 20 under the user's 52 vagina and the back insertion member 32 positioned in the user's 52 intergluteal cleft 54. The buttock engagement flaps 42 abut the user's 52 buttocks 56 to stabilize the back insertion member 32 in the intergluteal cleft 54. The buttock engagement flaps 42 may be retained against the user's 52 buttocks 56 by the undergarment 58 and additionally may frictionally engage the buttocks 56 so that the menstruation pad 10 resists movement with respect to the buttocks 56. The absorbent body 20 and the back insertion member 32 act to absorb exudate from the user's 52 body. The back insertion member 32 is particularly suited to absorbing exudate when the user 52 lies on her back, causing any exudate which is not immediately absorbed by the absorbent body 20 to flow toward the back portion 24 of the absorbent body 20 and into the intergluteal cleft 54. The menstruation pad 10 may be secured by adhering the base panel 12 to a crotch portion 60 of the undergarment 58. The wings 46 also may be folded around the crotch portion 60 of the undergarment 58 and adhered thereto.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A menstruation pad comprising:
a base panel being elongated between a front end and a back end, the base panel comprising a flexible material;
an absorbent body mounted on a top surface of the base panel, the absorbent body being elongated and being aligned parallel to the base panel, the absorbent body comprising an absorbent material, the absorbent body having a front portion, a back portion, and a middle portion;
a back insertion member mounted atop the back portion of the absorbent body, the back insertion member being elongated and being aligned parallel to the absorbent body, the back insertion member being sized and configured to fit into an intergluteal cleft of a user, the back insertion member comprising the absorbent material; and
a pair of buttock engagement flaps coupled to the base panel and positioned on opposite sides of the back insertion member, each buttock engagement flap comprising the flexible material, each buttock engagement flap having a thickness wherein the buttock engagement flap is configured to minimize visibility of an outline of the buttock engagement flap through an undergarment worn by the user.

2. The menstruation pad of claim 1, wherein the absorbent body narrows from each of the front portion and the back portion to the middle portion.

3. The menstruation pad of claim 1, wherein a forward end and a rearward end of the absorbent body are rounded.

4. The menstruation pad of claim 1, wherein the back insertion member is integrally formed with the absorbent body.

5. The menstruation pad of claim 1, wherein the back insertion member tapers from the absorbent body to a distal edge with respect to the absorbent body.

6. The menstruation pad of claim 1, wherein the back insertion member tapers from a central section of the back insertion member to each of a frontmost end and a backmost end of the back insertion member.

7. The menstruation pad of claim 1, wherein each buttock engagement flap is integrally formed with the base panel.

8. The menstruation pad of claim 1, wherein each buttock engagement flap tapers away from the base panel to a distal end with respect to the base panel.

9. The menstruation pad of claim 1, further comprising a pair of wings coupled to the base panel and positioned on opposite sides of the middle portion of the absorbent body, each wing comprising the flexible material.

10. The menstruation pad of claim 9, wherein each wing is integrally formed with the base panel.

11. The menstruation pad of claim 9, further comprising a plurality of adhesive layers configured to releasably attach to the undergarment, each adhesive layer being mounted on an associated one of the base panel and either wing of the pair of wings.

12. The menstruation pad of claim 1, further comprising an adhesive layer configured to releasably attach to the undergarment, the adhesive layer being mounted on the base panel.

13. A menstruation pad comprising:
a base panel being elongated between a front end and a back end, the base panel comprising a flexible material;
an absorbent body mounted on a top surface of the base panel, the absorbent body being elongated and being aligned parallel to the base panel, the absorbent body comprising an absorbent material, the absorbent body having a front portion, a back portion, and a middle portion, the absorbent body narrowing from each of the front portion and the back portion to the middle portion, a forward end and a rearward end of the absorbent body being rounded;
a back insertion member mounted atop the back portion of the absorbent body, the back insertion member being elongated and being aligned parallel to the absorbent body, the back insertion member being sized and configured to fit into an intergluteal cleft of a user, the back insertion member comprising the absorbent material, the back insertion member being integrally formed with the absorbent body, the back insertion member tapering from the absorbent body to a distal edge with respect to the absorbent body, the back insertion member tapering from a central section of the back insertion member to each of a frontmost end and a backmost end of the back insertion member;
a pair of buttock engagement flaps coupled to the base panel and positioned on opposite sides of the back insertion member, each buttock engagement flap comprising the flexible material, each buttock engagement flap having a thickness wherein the buttock engagement flap is configured to minimize visibility of an outline of the buttock engagement flap through an undergarment worn by the user, each buttock engagement flap being integrally formed with the base panel, each buttock engagement flap tapering away from the base panel to a distal end with respect to the base panel;
a pair of wings coupled to the base panel and positioned on opposite sides of the middle portion of the absorbent body, each wing comprising the flexible material, each wing being integrally formed with the base panel; and
a plurality of adhesive layers configured to releasably attach to the undergarment, each adhesive layer being mounted on an associated one of the base panel and either wing of the pair of wings.
